(19) United States
(12) Patent Application Publication
(10) Pub. No.: US 2006/0148008 A1
(43) Pub. Date: Jul. 6, 2006

(54) Method of characterizing dendritic cells

(76) Inventors: Derek Hart, Queensland (AU); Slavica Vuckovic, Queensland (AU)

Correspondence Address: Leopold Presser;Scully Scott Murphy & Presser, 400 Garden City Plaza, Ste 300, Garden City, NY, 11530, US

(21) Appl. No.: 10/524,578

(22) PCT Filed: Aug. 15, 2003
(86) PCT No.: PCT/AU03/01040

§ 371 Date: Mar. 1, 2006

(30) Foreign Application Priority Data
Aug. 15, 2002 (AU) 2002950778

(51) Int. Cl. G01N 33/567; C12N 5/08

(52) U.S. Cl. 435/7.21; 435/372

(57) ABSTRACT

A method of characterizing cells in a subclass of dendritic cells or precursors thereof in a biological sample. The method comprising placing the sample in contact with immunointeractive molecules directed to at least one dendritic cell immunogen (e.g. HLA-DR, CE123, CD11c, CD1b/c, BDCA2, BDCA3, BDCA4, Cb16, CD83, CD45, CD40, CMRF-44 AND CMRF-56) and CMRF-56) and at least one non-dendritic cell immunogen (e.g. CD3, CD19, CD14, CD56, CD11b, CD34) and detecting the presence of one or more dendritic cell immunogen-immunointeractive molecule complexes and the absence of one or more non-dendritic cell immunogen-immunointeractive molecule complexes. An analysis of the cells of the subclass of dendritic cells is performed based on the presence and absence of the above complexes. This method may be used in disease diagnosis by comparing the number of cells in the subset of dendritic cells or precursors thereof with the number of the same subset of cells found in an age-matched non-diseased patient.

### BACKGROUND OF THE INVENTION

1. Field of the Invention

The present invention relates generally to a method of characterizing cells in a subclass of dendritic cells and in particular identifying and enumerating the cells. The method of the present invention is useful for the diagnosis of diseases such as but not limited to cancer.

2. Description of the Prior Art

Dendritic cells belong to a family of antigen presenting cells and are characterized by the unique capability to activate immunologically naïve T cells. They are present in various tissue, and a variety of populations with dendritic cell characteristics have also been found in blood and non-lymphoid organs and are considered to be precursors of the dendritic cells in lymphoid tissue.

Characterization of dendritic cells in various tissue sites may be problematic, given that surface antigen expression, morphology and function may vary with the maturation and activation status of dendritic cells being examined in a given tissue.

It is also difficult to characterize dendritic cells in blood and tissues due to the lack of a single dendritic-specific antigen. The first method for enumeration of dendritic cells in blood was based on the expression of CMRF-44 and CD83 antigen which required overnight culture before detection and did not permit characterization of circulating blood dendritic cells. Other studies characterized dendritic cells by density centrifugation and/or negative selection. These procedures have not been satisfactory as they give low yield and purity of dendritic cells and are time consuming. Further, the manipulation can alter the cells functionally and does not allow for quantitation of dendritic cell frequency.

Accordingly, there is a need to develop improved methods of characterizing dendritic cells and in particular a sub-class of dendritic cells while minimizing the inclusion of cells other than the dendritic cells of interest.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In accordance with the present invention, a method of characterizing dendritic cells employing immunointeractive molecules directed to dendritic cell immunogens and non-dendritic cell immunogens has been developed.

The method involves characterizing cells in a subclass of dendritic cells or precursors thereof in a biological sample, said method comprising:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell immunogens;
(ii) detecting the presence of one or more dendritic cell immunogen-immunointeractive molecule complexes and the absence of one or more non-dendritic cell immunogen-immunointeractive molecule complexes;
(iii) performing an analysis of the cells of the subclass of dendritic cells based at least in part on the presence of one or more predetermined dendritic cell immunogen-immunointeractive molecule complexes and the absence of one or more predetermined non-dendritic cell immunogen-immunointeractive molecule complexes.
   wherein the presence of one or more predetermined dendritic cell immunogen-immunointeractive molecule complexes and the absence of one or more predetermined non-dendritic cell immunogen-immunointeractive molecule complexes characterizes a subclass of dendritic cells or precursors thereof.

Preferably, the one or more dendritic cell immunogens are selected from the group comprising HLA-DR, CD123, CD11c, CD1b/c BDCA2, BDCA3, BDCA4, CD16, CD83, CD45, CD40, CMRF-44 and CMRF-56 dendritic cell markers. Furthermore, the preferred immunointeractive molecules are antibodies.

The analysis generally further comprises:
(a) isolating dendritic cells or precursors thereof based at least in part on morphological characteristics to dendritic cells;
(b) isolating dendritic cells or precursors thereof based at least in part on the absence of expression of non-dendritic cell immunogens from the cells in the subclass created from step (a);
(c) isolating dendritic cells or precursors thereof based at least in part on the presence of expression of dendritic cell immunogens from the cells in the subclass created in step (a, b);
(d) optional steps of isolating cells or precursors thereof based in part on the presence of dendritic cell immunogens from previous subclasses (a, b, c);
(e) calculating the number of cells in the subclass of dendritic cells or precursors thereof created in step (c) or (d)

Also provided is a method of diagnosis by characterizing cells in a subclass of dendritic cells or precursors thereof in a biological sample, said method comprising:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell immunogens;
(ii) detecting the presence of one or more dendritic cell immunogen-immunointeractive molecule complexes and the absence of one or more non-dendritic cell immunogen-immunointeractive molecule complexes;
(iii) differentiating the subclass of dendritic cells based at least in part on the presence of one or more predetermined dendritic cell immunogen-immunointeractive molecule complexes and the absence of one or more predetermined non-dendritic cell immunogen-immunointeractive molecule complexes.
   wherein said differentiation further comprises:
(a) isolating dendritic cells or precursors thereof based at least in part on morphological characteristics to dendritic cells;
(b) isolating dendritic cells or precursors thereof based at least in part on the absence of expression of non-dendritic cell immunogens from the cells in the subclass created from step (a);
(c) isolating dendritic cells or precursors thereof based at least in part on the presence of expression of dendritic cell immunogens from the cells in the subclass created in step (a, b);
(d) optional steps of isolating cells or precursors thereof based in part on the presence of dendritic cell immunogens from previous subclasses (a, b, c);
(e) calculating the number of cells in the subclass of dendritic cells or precursors thereof created in step (c) or (d); and
(f) comparing the number of cells in the subset of dendritic cells or precursors thereof calculated from the biological sample to an acceptable number of cells found in an age-matched non-diseased patient.
   wherein the comparison of the number of cells in the subset of dendritic cells or precursors thereof calculated from the biological sample to an acceptable number of cells found in an age-matched non-diseased patient is a measure of the disease state.

Again, the preferred immunogens are HLA-DR, CD123, CD11c, CD1b/c, BDCA2, BDCA3, BDCA4, CD16, CD83, CD45, CD40, CMRF-44 and CMRF-56.

Hereinafter, reference to “dendritic cells” includes reference to precursors of dendritic cells. One particular subset identified by the method of the present invention includes a leucocyte population of CD45⁺CD40⁺CD11c⁻CD123⁻ cells which stimulate an allogenic T-cell response.

### BRIEF DESCRIPTION OF THE DRAWINGS

FIG. 1 is a graphical representation of the TruCOUNT (trademark) analysis of absolute numbers of blood dendritic cells. In FIG. 1a, a dot-plot demonstrates the side-scatter profile versus CD-45 expression. After gating for CD-45 expression (R1), Lin⁻HLA-DR⁺ dendritic cells (R2) were analyzed for their subclass composition according to their expression of CD11c (R3). In FIG. 1b, a dot plots also demonstrates the side-scatter profile versus CD-45 expression. After gating for CD-45 expression (R1), Lin⁻CD16⁺ dendritic cells (R2) were analyzed for their subclass composition according to their expression of CD11c (R3). In FIG. 1c, a dot plot also demonstrates the side-scatter profile versus CD-45 expression. After gating for CD-45 expression (R1), Lin⁻HLA-DR⁺ dendritic cells (R2) were analyzed for their subclass composition according to their expression of CD123 (R3).

FIG. 2 is a graphical representation of the comparison between TruCOUNT (trademark) analysis of absolute numbers of dendritic cells in adult blood and cord blood. The graphs provide a comparison between the numbers of dendritic cells (DC number/μg) in each CD11c⁺CD16⁻, CD11c⁺CD16⁺, and CD123^{hi} subset in adult blood and cord blood. A significant difference was established between CD123^{hi} dendritic cell counts in cord blood when compared to adult blood (P=0.0002), although no significant differences existed in the number of CD11c⁺CD16⁻, CD11c⁺CD16⁺ dendritic cells.

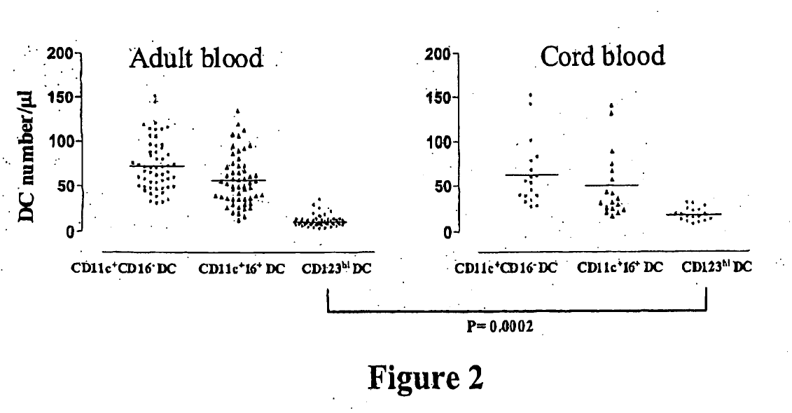

FIG. 3 is a graphical representation of a comparison between the numbers of dendritic cells (DC number/μl) in each CD11c⁺CD16⁻, CD11c⁺CD16⁺, and CD₁₂₃^{hi} subset in adult blood and cord blood obtained by single-platform (absolute DC counts were calculated using number of events in region containing DC population and number of beads, obtained by flow cytometry) versus dual-platform protocols (absolute DC counts were calculated using percentage of DC obtained by flow cytometry and mononuclear cell count obtained by an automated haematology analyzer). There were no significant differences between the mean dendritic cell number per μl of blood obtained by the two methods.

### DETAILED DESCRIPTION OF THE INVENTION

The present invention is predicated, in part, on the development of a method which can characterize one or more dendritic cell subclasses in a cell population. By combining the use of immunointeractive molecules directed to dendritic cell immunogens and immunointeractive molecules directed to non-dendritic cell immunogens, the inclusion of non-dendritic cells in a dendritic cell subclass is minimized. The method can also be used to diagnose diseases such as cancer and autoimmune diseases.

It must be noted that, as used in the subject specification, the singular forms “a”, “an” and “the” include plural aspects unless the context clearly dictates otherwise. Thus, for example, reference to an “immunogen” includes a single immunogen, as well as two or more immunogens; reference to “an immunointeractive molecule” includes a single active immunointeractive molecule, as well, as two or more immunointeractive molecules; and so forth.

Accordingly, the present invention provides a method of characterizing cells in a subclass of dendritic cells in a biological sample; said method comprising the steps of:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell immunogens;
(ii) detecting the presence of one or more dendritic cell immunogen-immunointeractive molecule complexes and the absence of one or more non-dendritic cell immunogen-immunointeractive molecule complexes;
(iii) performing an analysis of the cells in the subclass of dendritic cells based at least in part on the presence of one or more predetermined dendritic cell immunogen-immunointeractive molecule complexes and the absence of one or more predetermined non-dendritic cell immunogen-immunointeractive molecule complexes.
   wherein the presence of one or more predetermined dendritic cell immunogen-immunointeractive molecule complexes and the absence of one or more predetermined non-dendritic cell immunogen-immunointeractive molecule complexes characterizes a subclass of dendritic cells.

The term “characterizing” as used herein in relation to cells includes describing the distinguishing qualities of the cells. Included within this definition are the terms “identifying” and “enumerating”.

Reference to “dendritic cells” includes reference to cells exhibiting dendritic cell morphology, phenotype or functional activity and to mutants or variants thereof and to precursor cells of dendritic cells. The dendritic cells may be any developmental stage or state of differentiation. The morphological features of dendritic cells include, but are not limited to, long cytoplasmic processes, large cells with multiple fine dendrites (or other form of pseudopodia) or irregularly shaped membrane (although round cells are also observed). Phenotypic characteristics include, but are not limited to, expression of one or more of the murine and human CD11c,, CD123, MHC class I and II, CD1, CD4, CD8α, CD205 (Dec-205), 33D1, CD40, CD80, CD86, CD83, CD45, CMRF-44, CMRF-56, CD209 (DC-SIGN), CD208 (DC-LAMP), CD207 (Langerin) or CD206 (macrophage mannose receptor). Functional activity includes but is not limited to, a stimulatory capacity for naive allogenic T cells, the capacity to internalise antigens and re-expressing peptides of said antigens in association with MHC Class I and II molecules. The expression of particular morphological, phenotypic and functional features will vary according to the differentiative state of the dendritic cell. For example, dendritic cells precursors are known to be effective in taking up and presenting antigen. Expression of particular morphological, phenotypic and functional features may also vary between different populations of dendritic cells, such as dendritic cells arising from different cell lineages. For example, lymphoid-like dendritic cells vary from myeloid-like dendritic cells. “Variants” include, but are not limited to, cells exhibiting some but not all of the morphological or phenotypic features or functional activities of dendritic cells. “Mutants” include, but are not limited to, dendritic cells which are transgenic wherein said transgenic cells are engineered to express one or more genes such as genes encoding antigens, immune modulating agents, cytokines or receptors.

Reference herein to a “biological sample” includes reference to any sample of biological material derived from an animal such as, blood, for example, whole peripheral blood, cord blood, foetus blood, bone marrow, plasma, serum, urine, cultured cells, saliva or urethral swab, lymphoid tissues, for example tonsils, peyers patches, appendix, thymus. The biological sample which is tested according to the method of the present invention may be tested directly or may require some form of treatment prior to testing. For example, a biopsy sample may require homogenization to produce a cell suspension prior to testing. Furthermore, to the extent that the biological sample is not in liquid form (for example, it may be a solid, semi-solid or a dehydrated liquid sample), it may require the addition of a reagent, such as a buffer, to mobilize the sample. The mobilizing reagent may be mixed with the biological sample prior to placing the sample in contact with the one or more immunointeractive molecules or the reagent may be applied to the sample after the sample has been placed in contact with the one or more immunointeractive molecules.

The tern “animal” as used herein includes human, primate (gorilla, marmoset, macaque), livestock animal (e.g. sheep, pig, cow, horse, donkey), laboratory test animal (e.g. mouse, rat, rabbit, guinea pig), companion animal (e.g. dog, cat), captive wild animal (e.g. fox, kangaroo, deer), aves (e.g. chicken, geese, duck, emu, ostrich) reptile or fish or any other animal or bird which contains dendritic cells, and includes an embryonic or fetus of any of the aforementioned animals.

Reference to the term “predetermined” should be understood to mean determined prior to performing the method of the present invention. That is the person skilled in the art would have selected the one or more dendritic cell immunogen-immunointeractive molecule complexes and the one or more non-dendritic cell immunogen-immunointeractive molecule complexes upon which to base the analysis prior to performing the method.

The term “analysis” should be understood to mean an examination of the elements of a substance, that is, cells in the present invention. This term should be understood to encompass the qualitative and quantitative examination.

An “immunogen” is understood to be any substance which can elicit an immune response, for example an antigen. In the present invention, “dendritic cell immunogen” is an immunogen which is expressed on dendritic cells. These include and are not limited to murine and human CD11c, CD16, CD123, MHC class I and II, CD1, CD4, CD8α, CD205 (Dec-205), 33D1, CD40, CD80, CD86, CD83, CD45, CMRF-44, CMRF-56, -CD1b/c, BDCA2, BDCA3, BDCA4, CD209 (DC-SIGN), CD208(DC-LAMP), CD207 (Langerin) or CD206 (macrophage mannose receptor). Preferable human dendritic cell immunogens are HLA-DR, CD11c, CD16, CD123, CD1b/c, BDCA2, BDCA3 and BDCA4, CD83, CD45, CD40, CMRF-44 or CMRF-56.

Accordingly, this particular aspect of the present invention is directed to a method of characterizing cells of a subclass of dendritic cells thereof in a biological sample; said method comprising the steps of:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell immunogens;
(ii) detecting the presence of one or more dendritic cell immunogen-immunointeractive molecule complexes and the absence of one or more non-dendritic cell immunogen-immunointeractive molecule complexes;
(iii) performing an analysis of the cells in the subclass of dendritic cells based at least in part on the presence of one or more predetermined dendritic cell immunogen-immunointeractive molecule complexes and the absence of one or more predetermined non-dendritic cell immunogen-immunointeractive molecule complexes;
   wherein the presence of one or more predetermined dendritic cell immunogen-immunointeractive molecule complexes and the absence of one or more predetermined non-dendritic cell immunogen-immunointeractive molecule complexes characterizes a subclass of dendritic cells and the one or more dendritic cell immunogens are selected from the group comprising HLA-DR, CD123, CD11c, CD1a/b, BDCA3 and CD16, as well as immunogens from a population of CD11c⁻, CD123⁻ cells such as CD45⁺CD40⁺ cells.

Furthermore, “non-dendritic cell immunogen” should be understood to refer to an immunogen which is expressed on cells other than dendritic cells. For example, CD3 (T cells), CD19 (B cells), CD14 (monocytes), CD 56 (NK) and CD11b (granulocytes), CD34 (stem cells) are all immunogens expressed on leucocytes other than dendritic cells. It should be understood that dendritic cells, which lack these and other lineage immunogens, are designated Lin⁻. Dendritic cells which have low-side scatter characteristics, lack the aforementioned and other lineage associated immunogens and express HLA-DR are designated Lin⁻HLA-DR⁺.

Reference to “immunointeractive molecule” should be understood to be a reference to any molecule comprising an antigen binding portion or a derivative of said molecule. Examples of molecules contemplated by this aspect of the present invention include, but are not limited to, monoclonal and polyclonal antibodies (including synthetic antibodies), hybrid antibodies, humanized antibodies, catalytic antibodies) and T cell antigen binding molecules. Preferably, the immunointeractive molecule is an antibody.

Yet another aspect of the present invention, therefore, is directed to a method of characterizing cells of a subclass of dendritic cells in a biological sample; said method comprising the steps of:
(i) placing the sample in contact with one or more antibodies for a time and under conditions sufficient for one or more immunogen-antibody complexes to form, wherein said antibodies are directed against one or more dendritic cell immunogens and one or more non-dendritic cell immunogens;
(ii) detecting the presence of one or more dendritic cell immunogen-antibody complexes and the absence of one or more non-dendritic cell immunogen-antibody complexes;
(iii) performing an analysis of the cells in the subclass of dendritic cells based at least in part on the presence of one or more predetermined dendritic cell immunogen-antibody complexes and the absence of one or more predetermined non-dendritic cell immunogen-antibody complexes.
   wherein the presence of one or more predetermined dendritic cell immunogen-antibody molecule complexes and the absence of one or more predetermined non-dendritic cell immunogen-antibody molecule complexes characterizes a subclass of dendritic cells.

Reference to “placing the biological sample in contact” with one or more immunointeractive molecule should be understood as a reference to any method of facilitating the interaction of any one or more components of the biological sample with the one or more immunoreactive molecules such that coupling, binding or other association between the one or more components of the biological sample and the immunoreactive molecule may occur. In this instance, the one or more immunointeractive molecules were placed in contact with the sample simultaneously. Alternatively, the one or more immunointeractive molecules may be added in a staggered fashion.

Reference to an “immunogen-immunointeractive molecule complex” should be understood to mean that there is an interaction between an “immunogen” and an “immunointeractive molecule”. When the immunogen is an antigen and the immunointeractive molecule is an antibody, the forces which are involved in the interaction between an antibody and antigen in an antigen-antibody complex include electrostatic forces, hydrogen bonds, Van der Waals forces and hydrophobic bonds.

Reference to “detecting” the formation of the immunogen-immunointeractive molecule complex should be understood to encompass any method convenient which will be known to those skilled in the art. In the method of the invention exemplified herein, the immunointeractive molecules are labeled with fluorescent dyes and the fluorescence emissions from the immunogen-immunointeractive molecule complexes are detected by the photomultiplier tubes in a fluorescence-activated cell sorter (FACS). It should also be noted that alternative flow cytometry instruments with sorting facilities can also be used. The binding of the fluorescence labeled immunointeractive molecules to an immunogen indicates expression of the immunogen on a surface of a cell. The information can be presented on a histogram or on dot-plots. Alternatively, if large numbers of dendritic cells are required to be prepared, mechanical means of separation are preferable, for example, by exposing the dendritic cells to paramagnetic beads coated with an immunointeractive molecule that recognizes a particular immunogen or immunogen-immunointeractive molecule complex. The cells are then placed in a strong magnetic field; the cells attached to the beads are retained and the cells lacking the immunogen recognized by the immunointeractive molecule can be decanted off, leaving behind only the bound cells that express the immunogen of interest. The bound cells are positively selected for expression of the immunogen, while unbound cells are negatively selected. Another alterative detection technique is panning. Both the alternative techniques can also be used as a pre-enrichment step before sorting the cells by flow cytometry instruments.

Yet another aspect of the present invention is directed to a method of characterizing cells of a subclass of dendritic cells in a biological sample; said method comprising the steps of:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell-immunogens;
(ii) detecting the presence of one or more dendritic cell immunogen-immunointeractive molecule complexes and the absence of one or more non-dendritic cell immunogen-immunointeractive molecule complexes;
(iii) performing an analysis of the cells in the subclass of dendritic cells based at least in part on the presence of one or more predetermined dendritic cell immunogen-immunointeractive molecule complexes and the absence of one or more predetermined non-dendritic cell immunogen-immunointeractive molecule complexes;
   wherein the analysis in step (iii) further comprises:
(a) isolating dendritic cells based at least in part on morphological characteristics specific to dendritic cells;
(b) isolating dendritic cells based at least in part on the absence of expression of non-dendritic cell immunogens from the cells in the subclass created from step (a);
(c) isolating-dendritic cells based at least in part on the presence of expression of dendritic cell immunogens from the cells in the subclass created in step (a, b); and
(d) optional steps of isolating cells based in part on the presence of dendritic cell immunogens from previous subclasses (a, b, c).

Reference to “isolating” dendritic cells should be understood to refer to any method known to a persons skilled in the art of separating cells based on particular parameters in a given set of data. In the method of the invention exemplified herein, the method of detection of the immunointeractive-immunogen molecules is by FACS and, therefore, “electronic gating” was the chosen method of isolation. Electronic gating refers to data analysis which specifies cells (events) of interest. The electronic gates themselves are established in bivariate dot plots by drawing either a rectangle, eclipse or free-form encirclement around the population of interest. Cells can be gated according to “morphological characteristics”, as identified by forward and side scatter signals. Forward scatter is roughly proportional to the diameter of the cell, while side scatter is proportional to granularity. Typically, the person skilled in the art wishes to eliminate data from cell debris, dead cells and clumps of two or more cells. Dead cells have lower forward-scatter and higher side-scatter than living cells. Cells can also be gated on fluorescence signals to indicate expression of particular immunogen-immunointeractive molecule complexes.

Without limiting the present invention to any one mode of action, according to one embodiment, the cells from the sample are gated on the dot-plot CD45 versus SSC to exclude cell debris and dead cells but to include lymphocytes and monocytes. The resulting of subclass is designated R1. The next step is to gate on, R1 to create a subclass which is based in part on the lack of expression of lineage associated immunogens. The final step comprises gating on R2 to create a subclass based on predetermined dendritic cells immunogens.

Reference to “morphological characteristics” should be understood to refer to physical characteristics of cells such as size and granularity of cells.

Reference to “based at least in part” should be understood to mean that the parameter following this phrase should form a basis for isolation of dendritic cells. However, other parameters can also be used to form a basis for isolation.

Reference to “expression of dendritic cell immunogens” should be understood to be the appearance of a dendritic cell immunogen on the surface of cells following transcription and translation of the gene encoding the immunogen.

Reference to “expression of non-dendritic cell immunogens” should be understood to be the appearance of the non-dendritic cell immunogen on the surface of cells following transcription and translation of the gene encoding the immunogen.

Reference to “optional steps of isolating cells based in part on the presence of dendritic cell immunogens from previous subclasses” should be understood to mean that the person skilled in the art may wish to create further subclasses of dendritic cells from previous subclasses. The process of isolation of cells by, for example, electronic gating can be carried out on a subclass previously created.

Yet another aspect of the present invention is directed to a method of characterizing cells of a subclass of dendritic cells in a biological sample, said method comprising the steps of:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell-immunogens;
(ii) detecting the presence of one or more dendritic cell immunogen-immunointeractive molecule complexes and the absence of one or more non-dendritic cell immunogen-immunointeractive molecule complexes;
(iii) performing an analysis of the cells in the subclass of dendritic cells based at least in part on the presence of one or more predetermined dendritic cell immunogen-immunointeractive molecule complexes and the absence of one or more predetermined non-dendritic cell immunogen-immunointeractive molecule complexes;
   wherein the analysis in step (iii) further comprises:
(a) isolating dendritic cells based at least in part on morphological characteristics specific to dendritic cells;
(b) isolating dendritic cells based at least in part on the absence of expression of non-dendritic cell immunogens from the cells in the subclass created from step (a);
(c) isolating dendritic cells based at least in part on the presence of expression of dendritic cell immunogens from the cells in the subclass created in step (a, b);
(d) optional steps of isolating cells based in part on the presence of dendritic cell immunogens from previous subclasses (a, b, c); and
(e) calculating the number of cells in subclass of dendritic cells created in step (c) or (d).

Reference to the term “calculating” should be understood to mean to “ascertain or determine” by any method known to persons skilled in the art. For example, if a FACS is being used for the detection step of the method, it can analyze the data presented and provide the “number of cells positive for a particular parameter” or the “percentage of cells positive for a particular parameter”. Alternatively, if the TruCOUNT (trademark) Absolute Count Tubes are used to carry out the method of the present invention, which is true of the method exemplified, then the following formula may be used to calculate the absolute count of dendritic cells of a particular parameter:

$$\frac{\begin{matrix}number of events in region \\ containing dendritic cell subclass\end{matrix}}{number of beads counted} \times \frac{\begin{matrix}number of beads per \\ {TruCOUNT}^{TM} tube\end{matrix}}{test volume}$$

Yet another aspect of the instant invention is directed to a method of diagnosing disease by characterizing cells of a subclass of dendritic cells in a biological sample; said method comprising the steps of:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell-immunogens;
(ii) detecting the presence of one or more dendritic cell immunogen-immunointeractive molecule complexes and the absence of one or more non-dendritic cell immunogen-immunointeractive molecule complexes;
(iii) differentiating the subclass of dendritic cells based at least in part on the presence of one or more predetermined dendritic cell immunogen-immunointeractive molecule complexes and the absence of one or more predetermined non-dendritic cell immunogen-immunointeractive molecule complexes.
   wherein differentiating the subclass in step (iii) further comprises:
(f) isolating dendritic cells based at least in part on morphological characteristics to dendritic cells;
(g) isolating dendritic cells based at least in part on the absence of expression of non-dendritic cell immunogens from the cells in the subclass created from step (a);
(h) isolating dendritic cells based at least in part on the presence of expression of dendritic cell immunogens from the cells in the subclass created in step (a, b);
(i) optional steps of isolating cells based in part on the presence of dendritic cell immunogens from previous subclasses (a, b, c);
(j) calculating the number of cells in the subclass of dendritic cells created in step (c) or (d); and
(k) comparing the number of cells in the subset of dendritic cells calculated from the biological sample to an acceptable number of cells found in an age-matched non-diseased patient.
   wherein the comparison of the number of cells in the subset of dendritic cells calculated from the biological sample to an acceptable number of cells found in an age-matched non-diseased patient is a measure of the disease state.

As stated previously, the preferred immunointeractive molecule of the present invention is an antibody.

Accordingly, yet another aspect of the instant invention is directed to a method of diagnosing disease by characterizing cells of a subclass of dendritic cells in a biological sample; said method comprising the steps of:
(iv) placing the sample in contact with one or more antibodies molecules for a time and under conditions sufficient for one or more immunogen-antibody molecule complexes to form, wherein said antibody is directed against one or more dendritic cell immunogens and one or more non-dendritic cell immunogens;
(v) detecting the presence of one or more dendritic cell immunogen-antibody complexes and the absence of one or more non-dendritic cell immunogen-antibody molecule complexes;
(vi) differentiating the subclass of dendritic cells based at least in part on the presence of one or more predetermined dendritic cell immunogen-antibody molecule complexes and the absence of one or more predetermined non-dendritic cell immunogen-antibody molecule complexes.
   wherein differentiating the subclass in step (iii) further comprises:
(l) isolating dendritic cells based at least in part on morphological characteristics to dendritic cells;
(m) isolating dendritic cells based at least in part on the absence of expression of non-dendritic cell immunogens from the cells in the subclass created from step (a);
(n) isolating dendritic cells based at least in part on the presence of expression of dendritic cell immunogens from the cells in the subclass created in step (a, b);
(o) optional steps of isolating cells based in part on the presence of dendritic cell immunogens from previous subclasses (a, b, c);
(p) calculating the number of cells in the subclass of dendritic cells created in step (c) or (d); and
(q) comparing the number of cells in the subset of dendritic cells calculated from the biological sample to an acceptable number of cells found in an age-matched non-diseased patient.
   wherein the comparison of the number of cells in the subset of dendritic cells calculated from the biological sample to an acceptable number of cells found in an age-matched non-diseased patient is a measure of the disease state.

For example, the method of obtaining this relevant information may be applied to cancer, HIV, autoimmune diseases (e.g. diabetes) and transplant recipients.

The present invention further extends to an isolated population of CD11c⁻CD123⁻ CD45⁺CD40⁺ leucocytes which stimulate an allogenic T-cell response.

Although not wishing to limit the invention to any one theory or mode of action, it is proposed that this population of cells is a population of DC precursor cells.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

The present invention is further described by the following non-limiting Examples.

### EXAMPLE 1

### Blood Specimens

Normal adult blood samples (n=55) were collected from normal adult donors, according to Ethical Committee Guidelines, with appropriate informed consent. Umbilical cord blood samples (n=18) were collected from full-term deliveries with informed consent from the mothers and approval of the institutional ethics committee. Adult and cord blood specimens were collected in EDTA Vacutainer (K2E) and processed within 8 hours of collection. Blood cell counts were performed on an Technicon H.3-RTX (Bayer) automated haematology analyzer.

### EXAMPLE2

### Specimen Staining Procedure And Sample Acquisition

Except where indicated, all chemicals were purchased from Becton Dickinson (BDIS, Sydney, Australia). The assay is designed to detect three different subsets of blood dendritic cells (DC), CD11c⁺CD16⁻ DC, CD11c⁺CD16⁺ DC and CD123^{hi} DC in each specimen sample. Assay was performed in duplicate for each DC subset. For each specimen sample, three TruCOUNT (trademark) tubes were labeled as follows: CD11c⁺ CD16⁻ DC #1, CD11c⁺CD16⁺ DC #2 and CD123^{hi} DC #3. Directly conjugated monoclonal antibodies (mAb) were added to TruCOUNT (trademark) tubes as follows: CD11c⁺CD16⁻ DC #1, (Lin cocktail CD3/19/14/56/16/34-Fluorescein Isothiocyanate [FITC], CD11c-Phycoerythrin [PE], HLA-DR-Allophycocyanin [APC], CD45-Peridinin Chlorophyll Protein [PerCP]), CD11c⁺CD16⁺ DC #2 (Lin cocktail CD3/19/14/56/34-FITC, CD16-PE, CD11c-APC, CD45-PerCP and CD123^{hi} DC #3 (Lin cocktail CD3/19/14/56/16/34-FITC, CD123-PE, HLA-DR-APC, CD45-PerCP). 50 μl of well-mixed EDTA treated whole blood was added by reverse pipetting technique to each TruCOUNT (trademark) tube. Tubes were vortexed gently and incubated for 15 minutes in the dark at room temperature. 450 μl of FACS Lysing Solution was added to each tube, vortexed gently and incubated for 15 minutes in the dark at room temperature. Samples were vortexed gently and analyed within 1-2 hours. Data were collected on a FACSCalibur (BDIS, San Jose, Calif.) equipped with a second red-diode laser operating at 635 nm.

Control samples from normal adults were run daily to optimise instrument setting and as a quality control check of the system. In dot plots displaying CD45 versus SSC, the FL3 threshold was positioned to minimise debris and ensure populations of white nuclear cells were included. Control tubes containing the same amount of isotype control mAb were used for setting the fluorescence compensation. For the FACSCalibur, the compensation settings for double-laser, four color analysis where: FL1-% FL2, 1.8; FL2-% FL1, 14.2; FL2-% FL3, 0.0; FL3-% FL2, 10.0; FL3-% FL4, 0.7; FL4-% FL3, 14.8; threshold FL3 value 365. Acquisition times per tube ranged from 8-9 minutes using “lyse-no-wash protocol”. A total of 20000-25000 bead events per tube were collected.

### EXAMPLE3

### Gating Strategies And Sample Analysis

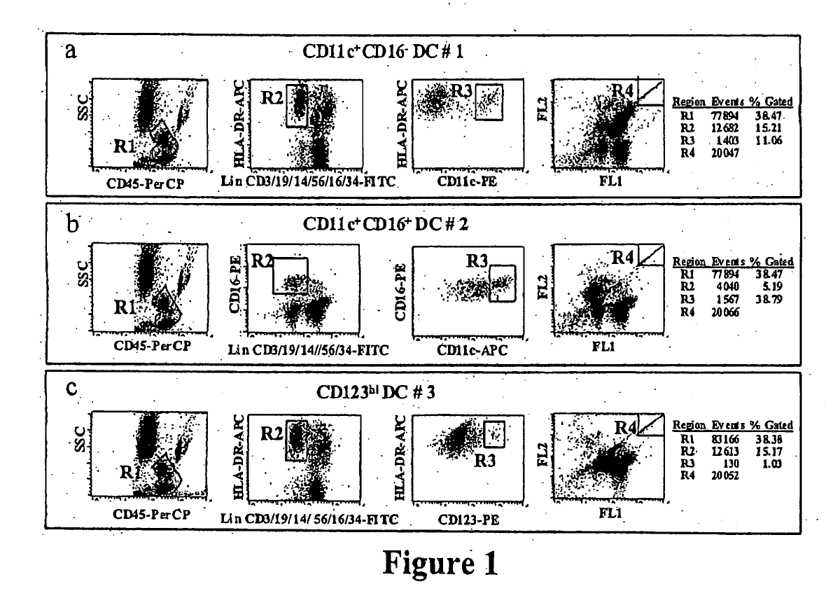

Data were analyzed using the CellQuest 3.1 f software. Multi-gating strategy applied for the TruCOUNT (trademark) tube CD11c⁺ CD16⁻ DC #1, CD11c⁺CD16⁺ DC #2 and CD123^{hi} DC #3. shown in FIG. 1. For the tube CD11c⁺ CD16⁻ DC #1 (FIG. 1a), Region 1 (R1) was created to include all CD45⁺ lymphocytes and monocytes and eliminate platelets, red blood cells, debris and aggregates. Events from region R1 were displayed in a dot-plot Lin versus HLA-DR and R2 was created to include all Lin⁻HLA-DR⁺ events. Events from R2 were displayed in a dot-plot CD11c versus HLA-DR and R3 was created to include all CD11c⁺CD16⁻ DC. For the tube CD11c⁺ CD16⁺ DC #2 (FIG. 1b) events from region R1 were displayed in a dot-plot Lin versus CD16 and R2 was created to include all Lin⁻CD16⁺ events. Events from R2 were displayed in a dot-plot CD11c versus CD16 and R3 was created to include all CD11c⁺CD16⁺ DC. For tube CD123^{hi} DC #3 (FIG. 1c), events from region R1 were displayed in a dot-plot Lin versus HLA-DR and R2 was created to include all Lin⁻HLA-DR⁺ events. Events from R2 were displayed in a dot-plot CD123^{hi} versus HLA-DR⁺, and R3 was created to include all CD₁₂₃^{hi} DC. TruCOUNT (trademark) beads, evident as a population of bright events in each fluorescence (FL) channel, were enumerated in region R4 in an ungated dot-plot FL1 versus FL2 (FIGS. 1a, 1b, 1c). Region R4 was drawn to include the last channel of the FL1 Log (on the right) and FL2 Log scales (on the top) and tightly around the beads to exclude any non-bead events.

### EXAMPLE 4

### DC Enumeration: Single-Platform TruCOUNT (Trademark) Protocol

The number of DC per μl blood was calculated by the following formula: number of events in region containing DC population (R3, FIGS. 1a, 1b, 1c)/number of beads counted (R4)×number of beads per TruCOUNT (trademark) tube blood volume.

### EXAMPLE 5

### DC Enumeration: Dual-Platform Protocol

The number of DC per μl of blood was calculated by using the percentage of DC in R1 (FIGS. 1a, 1b, 1c) determined by flow cytometry and the mononuclear cell count determined by an automated hematology analyzer.

### EXAMPLE 6

### Statistical Analysis

Comparison of the number of DC obtained in adult blood and cord blood samples was determined by unpaired t-test. Significance was defined as P<0.05. Within-sample reproducibility and the correlation between the two methods was determined by using a multilevel model. (SAS Proc Mixed).

### EXAMPLE 7

### Quality Control

Control blood samples were run daily before test sample, as a quality control check of the system. Lymphocyte, monocyte and granulocyte populations were visually inspected as compact, distinct clusters in a dot-plot CD45 versus SSC (FIG. 1). Clear separation between clusters was routinely observed. Lymphocyte or monocyte counts calculated by the single-platform TruCOUNT (trademark) protocol with compared with counts determined by an automated haematology analyzer. There were no significant differences between the mean monocyte or lymphocyte counts per μl of blood obtained by these two methods (Table 1). The two methods showed good correlation, indicating that system setting allowed precise measurement of blood mononuclear cells in whole blood samples.

TABLE 1
Lymphocyte and monocyte counts analyzed by
the single-platform TruCOUNT (trademark) protocol
versus automated hematology analyzer

| | | | TRUCOUNT | BLOOD ANALYZER |
|---|---|---|---|---|
| SAMPLE | N | CELLS | MEAN ± SD | MEAN ± SD |
| Adult | 70 | Lymphocytes | 2097 ± 551 | 2073 ± 547 |
| blood | 70 | Monocytes | 379 ± 99 | 370 ± 101 |
| Cord | 27 | Lymphocytes | 5210 ± 1078 | 5619 ± 1658 |
| Blood | 28 | Monocytes | 1249 ± 617 | 1267 ± 557 |

### EXAMPLE 8

### Enumeration of Blood DC Subsets Using Single-Platform TruCOUNT (Trademark) Protocol

The no-wash single-platform TruCOUNT (trademark) protocol was applied to enumerate blood CD11c⁺CD16⁻ DC, CD11c⁺CD16⁺ DC, and CD123^{hi} DC per μl of normal adult blood and cord blood. By this method, in adult blood absolute CD11c⁺ CD16⁻ DC counts were 73±4/μl, CD11c⁺CD16⁺ DC counts were 57±4/μl, and CD123^{hi}DC counts were 10±1/μl (mean±SD, n=55). In cord blood (n=18) compared to adult blood, no significant differences in the number of CD11c⁺CD16⁻DC and CD11c⁺CD16⁺DC (mean±SD, 62±9/μl, P=0.2078; 49±9/μl, P=0.3866, respectively), but significantly higher numbers of CD123^{hi} DC (17±2μl, P<0.002), were detected. Each TruCOUNT (trademark) tube CD11c⁺CD16⁻ DC #1, CD11c⁺CD16⁻ DC #2 and CD123^{hi} DC #3 was performed in duplicate to assess within-sample reproducibility. A high level of reproducibility was achieved giving within-sample variations in the range 1%-3%. Absolute numbers of blood DC obtained by this single-platform TruCOUNT protocol were higher than previously reported likely because this assay eliminates lots of DC during cell culture, centrifugation, and washing procedures. Also single-platform TruCOUNT protocol eliminates sources of variability associated with the dual-platform calculation protocol.

All together, CD11c⁺CD16⁻ DC, CD11c⁺CD16⁺ DC and CD123^{hi} DC in adult blood account for 40%, and in cord blood for 14%, of total Lin⁻HLA-DR⁺ events (FIG. 1). It was found that 211±115/μl (range −16-536) in adult blood and 813±309/μl (range 263-1363) events in cord blood meeting the light scatter (low SSC) and fluorescence criteria (Lin⁻HLA-DR⁺) of blood DC, were not accounted for by the analyzed blood DC subsets. Phenotype and function of this blood population are as yet described and more stringent scrutiny is needed in order to determine these parameters for normal and disease conditions.

### EXAMPLE 9

### Comparison of Single-Platform TruCOUNT (Trademark) And Dual-Platform DC Counting Protocols

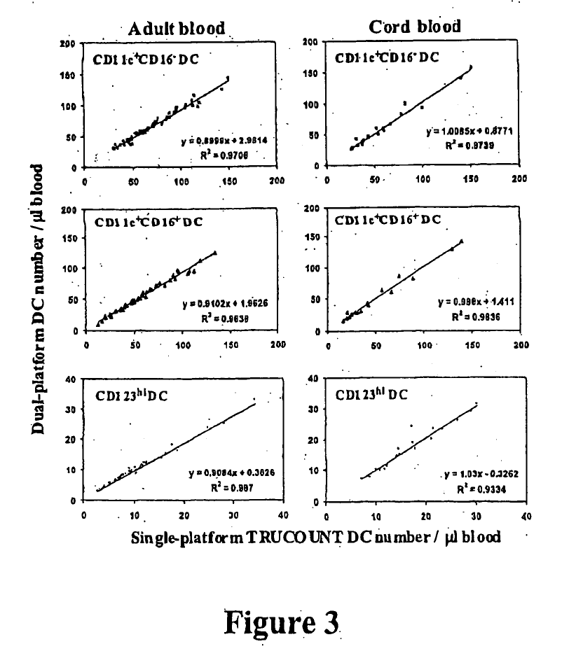

Absolute DC number per μl of blood was analyzed simultaneously by the single-platform TruCOUNT (trademark) protocol and the dual-platform protocol in both whole adult blood and cord blood samples. The two protocols showed good correlation (FIG. 3). There were no significant differences between the mean DC number per μl of blood obtained by the two methods (FIG. 3).

Those skilled in the art will appreciate that the invention described herein is susceptible to variations and modifications other than those specifically described. It is to be understood that the invention also includes all the steps, features, compositions and compounds referred to or indicated in the specification, individually or collectively and any and all combinations of any two or more of said steps or features.

1. A method of characterizing cells in a subclass of dendritic cells or precursors thereof in a biological sample, said method comprising:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell immunogens;
(ii) detecting the presence of one or more dendritic cell immunogen-immunointeractive molecule complexes and the absence of one or more non-dendritic cell immunogen-immunointeractive molecule complexes;
(iii) performing an analysis of the cells of the subclass of dendritic cells based at least in part on the presence of one or more predetermined dendritic cell immunogen-immunointeractive molecule complexes and the absence of one or more predetermined non-dendritic cell immunogen-immunointeractive molecule complexes.
wherein the presence of one or more predetermined dendritic cell immunogen-immunointeractive molecule complexes and the absence of one or more predetermined non-dendritic cell immunogen-immunointeractive molecule complexes characterizes a subclass of dendritic cells or precursors thereof.

2. A method according to claim 1 wherein performing the analysis of cells further comprises:
(a) isolating dendritic cells or precursors thereof based at least in part on morphological characteristics to dendritic cells;
(b) isolating dendritic cells or precursors thereof based at least in part on the absence of expression of non-dendritic cell immunogens from the cells in the subclass created from step (a);
(c) isolating dendritic cells or precursors thereof based at least in part on the presence of expression of dendritic cell immunogens from the cells in the subclass created in step (a, b);
(d) optional steps of isolating cells or precursors thereof based in part on the presence of dendritic cell immunogens from previous subclasses (a, b, c);
(e) calculating the number of cells in the subclass of dendritic cells or precursors thereof created in step (c) or (d)

3. A method of diagnosis by characterizing cells in a subclass of dendritic cells or precursors thereof in a biological sample, said method comprising:
(i) placing the sample in contact with one or more immunointeractive molecules for a time and under conditions sufficient for one or more immunogen-immunointeractive molecule complexes to form, wherein said immunointeractive molecules are directed against one or more dendritic cell immunogens and one or more non-dendritic cell immunogens;
(ii) detecting the presence of one or more dendritic cell immunogen-immunointeractive molecule complexes and the absence of one or more non-dendritic cell immunogen-immunointeractive molecule complexes;
(iii) differentiating the subclass of dendritic cells based at least in part on the presence of one or more predetermined dendritic cell immunogen-immunointeractive molecule complexes and the absence of one or more predetermined non-dendritic cell immunogen-immunointeractive molecule complexes.
wherein said differentiation further comprises:
(a) isolating dendritic cells or precursors thereof based at least in part on morphological characteristics to dendritic cells;
(b) isolating dendritic cells or precursors thereof based at least in part on the absence of expression of non-dendritic cell immunogens from the cells in the subclass created from step (a);
(c) isolating dendritic cells or precursors thereof based at least in part on the presence of expression of dendritic cell immunogens from the cells in the subclass created in step (a, b);
(d) optional steps of isolating cells based in part on the presence of dendritic cell immunogens from previous subclasses (a, b, c);
(e) calculating the number of cells in the subclass of dendritic cells or precursors thereof created in step (c) or (d); and
(f) comparing the number of cells in the subset of dendritic cells or precursors thereof calculated from the biological sample to an acceptable number of cells found in an age-matched non-diseased patient.
wherein the comparison of the number of cells in the subset of dendritic cells calculated from the biological sample to an acceptable number of cells found in an age-matched non-diseased patient is a measure of the disease state.

4. A method according to claim 1 to 3 wherein the biological sample is derived from an animal.

5. A method according to claim 4 wherein the animal is a human.

6. A method according to claim 5 wherein the immunointeractive molecule is an antibody or functional equivalent thereof.

7. A method according to claim 6 wherein the functional equivalent is a derivative, fragment, homolog, analog or chemical equivalent of the antibody.

8. A method according to claim 5 wherein the one or more dendritic cell immunogens are selected from the group comprising HLA-DR, CD123, CD11c, CD1b/c BDCA2, BDCA3, BDCA4, CD16, CD83, CD45, CD40, CMRF-44 and CMRF-56.

9. A method according to claim 5 wherein the dendritic cell immunogen is HLA-DR.

10. A method according to claim 5 wherein the dendritic cell immunogen is CD123.

11. A method according to claim 5 wherein the dendritic cell immunogen is CD11c.

12. A method according to claim 5 wherein the dendritic cell immunogen is CD1b/c.

13. A method according to claim 5 wherein the dendritic cell immunogen is BDCA2.

14. A method according to claim 5 wherein the dendritic cell immunogen is BDCA3.

15. A method according to claim 5 wherein the dendritic cell immunogen is BDCA4.

16. A method according to claim 5 wherein the dendritic cell immunogen is CD16.

17. A method according to claim 5 wherein the dendritic cell immunogen is CD83.

18. A method according to claim 5 wherein the dendritic cell immunogen is CD45.

19. A method according to claim 5 wherein the dendritic cell immunogen is CD40.

20. A method according to claim 5 wherein the dendritic cell immunogen is CMRF-44.

21. A method according to claim 5 wherein the dendritic cell immunogen is CMRF-56.

22. A method according to claim 5 wherein the one or more non-dendritic cell immunogens are selected from the group comprising CD3, CD19, CD14, CD56, CD11b and CD34.

23. A method according to claim 5 wherein the non-dendritic cell immunogen is CD3.

24. A method according to claim 5 wherein the non-dendritic cell immunogen is CD19.

25. A method according to claim 5 wherein the non-dendritic cell immunogen is CD14.

26. A method according to claim 5 wherein the non-dendritic cell immunogen is CD56.

27. A method according to claim 5 wherein the non-dendritic cell immunogen is CD11b.

28. A method according to claim 5 wherein the non-dendritic cell immunogen is CD34.

29. A leukocyte population isolated according to the method of claims 1 to 28 that is CD45⁺CD40⁺CD11c⁻CD123⁻.
